(19) United States
(12) Patent Application Publication
(10) Pub. No.: US 2025/0002990 A1
(43) Pub. Date: Jan. 2, 2025

(54) DIRECT SEQUENCING BIOMOLECULES AND MODIFICATIONS THEREOF WITH TUNNELING ENHANCED OPTICAL SPECTROSCOPY ON NANOPORE CHIP

(71) Applicants: ARIZONA BOARD OF REGENTS ON BEHALF OF ARIZONA STATE UNIVERSITY, Scottsdale, AZ (US); ELECULAR LLC, Scottsdale, AZ (US)

(72) Inventors: Quan QING, Chandler, AZ (US); Peiming ZHANG, Gilbert, AZ (US)

(21) Appl. No.: 18/709,791

(22) PCT Filed: Nov. 14, 2022
(86) PCT No.: PCT/US2022/049816

§ 371 Date: May 13, 2024

Related U.S. Application Data
(60) Provisional application No. 63/279,469, filed on Nov. 15, 2021.

(51) Int. Cl. C12Q 1/6869; G16B 30/00; G16B 40/10

(52) U.S. Cl. CPC C12Q 1/6869; G16B 30/00; G16B 40/10

(57) ABSTRACT

Disclosed are a nanopore-optical electronic device and a method for sequencing a biomolecule and modifications thereof. The nanopore-optical electronic device includes: a cis-fluidic chamber and a trans-fluidic chamber fabricated in a planar substrate; a nano-fluidic channel in between and connecting the cis-fluidic chamber and the trans-fluidic chamber; a first electrode and a second electrode sealed in the nano-fluidic channel and forming a nanogap/nanopore therebetween; a third electrode and fourth electrode disposed in the cis-fluidic chamber and the trans-fluidic chamber, respectively; an optical coupling element coupling an electromagnetic beam with the nanogap; an optical detector detecting an optical signal from the nanogap when a biomolecule translocates through the nanopore; and a current-measuring circuit concurrently measuring a tunneling current and ionic current when the biomolecule translocates through the nanopore. The recorded optical signal and tunneling current and ionic current signals are coincidental in time and colocalized in space.

### CROSS REFERENCE TO RELATED APPLICATION

The present application claims priority to and the benefit of U.S. Provisional Application No. 63/279,469, filed on Nov. 15, 2021, the entire contents of which being incorporated herein by reference in its entirety.

### STATEMENT OF GOVERNMENT INTEREST

The present application was made with government support under FA9550-16-1-0052 awarded by USAFR/AFOSR. The government has certain rights in the invention.

### TECHNICAL FIELD

The present disclosure relates to the field of molecule detection, in particular to a nanopore-optical electronic device and a method for sequencing a biomolecule and modifications thereof.

### BACKGROUND

There are more than 140 known modified ribonucleotides in RNome, which influence structures and functions of ribonucleic acid (RNA). For example, modifications together with splicing can change RNA topology and chemical property, which controls how nucleic acids regulate cellular and organismal functions. Defects in the modifications can lead to multiorgan failures, cancers, and neurological disorders. In addition, the accurate knowledge of modifications of RNA genomes is also very valuable for the early identification and control of RNA virus outbreaks, as well as for the development of RNA-based therapeutics including mRNA vaccines, because the sequence information from the complementary deoxyribonucleic acid (cDNA) can only provide a rough guidance.

However, how these modifications are distributed in RNA transcripts remains unknown. Next-generation deoxyribonucleic acid (DNA) sequencing technology determines an RNA sequence by reverse transcription of RNA to cDNA, which results in a loss of the position and identity of RNA modifications. Although a protein nanopore sequencer has been very successful in rapid single molecule DNA sequencing with long read length, and can potentially sequence RNA directly, it lacks a single-nucleotide resolution, which makes identifying modified ribonucleotides extremely challenging, if not impossible.

Furthermore, proteins and peptides are composed of 20 amino acids and may have even more modifications. Conventional nanopore technologies would not have the capacity to decode these variations based on solely ionic current readouts, and methods to sequence proteins/peptides in the art have very limited bandwidth and/or specificity.

Therefore, there is an urgent need for an effective technique to determine the identity and position of all modifications in full-length biomolecules at single molecule and/or transcriptome-wide scales.

### SUMMARY

The present disclosure is directed to a nanopore-optical electronic device and a method for sequencing a biomolecule and modifications thereof.

According to a first aspect of embodiments of the present disclosure, a nanopore-optical electronic device is provided for sequencing a biomolecule. The nanopore-optical electronic device comprises:

a cis-fluidic chamber and a trans-fluidic chamber in a planar substrate; a nano-fluidic channel connecting the cis-fluidic chamber and the trans-fluidic chamber;

a first electrode and second electrode in the nano-fluidic channel, the first electrode and the second electrode forming a nanogap between the first electrode and the second electrode;

a third electrode and fourth electrode in the cis-fluidic chamber and the trans-fluidic chamber, respectively;

an optical coupling element configured to couple an electromagnetic beam with the nanogap;

an optical detector configured to detect an optical signal from the nanogap when a biomolecule translocates through the nanogap; and

a current-measuring circuit configured to measure a tunneling current between the first electrode and the second electrode and an ionic current between the third electrode and the fourth electrode.

In some embodiments, the planar substrate may be a transparent substrate, such as glass or quartz.

In some embodiments, the planar substrate may be a non-transparent substrate, such as silicon coated with a layer of oxide.

In some embodiments, the first electrode, second electrode, third electrode, and fourth electrode may be formed of gold, palladium, platinum, silver, or combinations thereof.

In some embodiments, the first and second electrodes may be electrochemically deposited with one or more metal materials within the nano-fluidic channel and under feed-back control, thereby forming the nanogap with a single path for the biomolecule to translocate from the cis-fluidic chamber to the trans-fluidic chamber, wherein a distance between the first and second electrodes is between 1 nm and 100 nm, the nanogap is self-aligned with the first and second electrodes and has a narrowest bottleneck in the path between the cis-fluidic and trans-fluidic chambers.

In some embodiments, the first and second electrodes being electrochemically deposited with one or more metal materials within the nano-fluidic channel and under feed-back control may include a pulsed electrochemical deposition with a pulse width of 50 ms or less and a rest period of about 2 seconds between pulses.

In some embodiments, the pulse width may be in a range of 2 ms to 5 ms; in some embodiments, the pulse width may be less than 2 ms but greater than 1 μs.

In some embodiments, the distance between the first and second electrodes may be in a range of 1 nm to 10 nm.

In some embodiments, the first electrode and the second electrode are orthogonal to the nano-fluidic channel and forms a self-aligned transverse tunneling junction with the nanogap on the planar substrate.

In some embodiments, the one or more mental materials may include, but are not limited to, silver (Ag), nickel (Ni), cobalt (Co), Ni alloy, Co alloy, gold, palladium, platinum, iridium, or alloys thereof, or combinations thereof.

In some embodiments, the electromagnetic beam may be a laser beam.

In some embodiments, the laser beam may be an ultraviolet laser beam; in some embodiments, the laser beam may be a visible laser beam; in some embodiments, the laser beam may be an infrared laser beam.

In some embodiments, the optical coupling element may include a lens assembly, a fiber optical coupling element, a waveguide element, or a combination thereof.

In some embodiments, the optical coupling element may include a lens assembly.

In some embodiments, the optical coupling element may include a fiber optical coupling element such as a fiber-in-fiber-out coupling element.

In some embodiments, the optical coupling element may include a waveguide element.

In some embodiments, the optical coupling element may further include a polarizer.

In some embodiments, the optical signal is selected from the group consisting of a fluorescence or phosphorescence signal, a tip-enhanced Raman signal, a surface-enhanced Raman signal, and a combination thereof.

In some embodiments, the optical signal is a fluorescence or phosphorescence signal.

In some embodiments, the optical signal is a tip-enhanced Raman signal.

In some embodiments, the optical signal is a surface-enhanced Raman signal.

In some embodiments, the first electrode and second electrode may include an oxide layer such as a metal oxide layer.

In some embodiments, the first electrode and second electrode may include a surface modification layer including, but not limited to, polyethylene glycol thiol (PEG-thiol), alkyl thiol, cysteine, 4(5)-(2-mercaptoethyl)-1H-imidazole-2-carboxamide, or a combination thereof.

In some embodiments, the first electrode and second electrode may include a reader molecule, such as cysteine, 4(5)-(2-mercaptoethyl)-1H-imidazole-2-carboxamide.

In some embodiments, the biomolecule may include, but is not limited to, RNA, DNA, protein, peptide.

In some embodiments, the biomolecule may be RNA.

In some embodiments, the biomolecule may be DNA.

In some embodiments, the biomolecule may be peptide or protein.

In some embodiments, the tunneling current, ionic current, and optical signal are coincidental in time and colocalized in space, and originate from the same biomolecule that translocates the nanogap.

According to a second aspect of embodiments of the present disclosure, a method for sequencing a biomolecule is provided, the method comprising:

providing a nanopore-optical electronic device including:
a cis-fluidic chamber and a trans-fluidic chamber in a planar substrate,
a nano-fluidic channel connecting the cis-fluidic chamber and the trans-fluidic chamber,
a first electrode and a second electrode in the nano-fluidic channel, the first electrode and the second electrode forming a nanogap between the first electrode and the second electrode,
a third electrode and a fourth electrode in the cis-fluidic chamber and the trans-fluidic chamber, respectively,
an optical coupling element configured to couple an electromagnetic beam with the nanogap,
an optical detector configured to detect an optical signal from the nanogap when a biomolecule translocates through the nanogap, and

a current-measuring circuit configured to measure a tunneling current between the first electrode and the second electrode and an ionic current between the third electrode and the fourth electrode;

providing a sample solution including a biomolecule in the cis-fluidic chamber;

providing a first bias between the third electrode and the fourth electrode across the nano-fluidic channel;

providing a second bias across the first electrode and the second electrode across the nanogap;

concurrently measuring:
a tunneling current between the first electrode and the second electrode,
an ionic current between the third electrode and the fourth electrode when the biomolecule translocates through the nanopore, and
an optical signal from the nanogap when the biomolecule translocates through the nanopore; and

correlating the tunneling current, the ionic current, and the optical signal to determine a sequence of the biomolecule.

In some embodiments, the planar substrate may be a transparent substrate, such as glass or quartz.

In some embodiments, the planar substrate may be a non-transparent substrate, such as silicon coated with a layer of oxide.

In some embodiments, the first electrode, second electrode, third electrode, and fourth electrode may be formed of gold, palladium, platinum, silver, or combinations thereof.

In some embodiments, the first and second electrodes may be electrochemically deposited with one or more metal materials within the nano-fluidic channel and under feed-back control, thereby forming the nanogap with a single path for the biomolecule to translocate from the cis-fluidic chamber to the trans-fluidic chamber, wherein a distance between the first and second electrodes is between 1 and 100 nm, and the nanogap is self-aligned with the first and second electrodes and has a narrowest bottleneck in the path between the cis-fluidic and trans-fluidic chambers.

In some embodiments, the first and second electrodes being electrochemically deposited with one or more metal materials within the nano-fluidic channel and under feed-back control may include a pulsed electrochemical deposition with a pulse width of 50 ms or less and a rest period of about 2 seconds between pulses.

In some embodiments, the pulse width may be in a range of 2 ms to 5 ms; in some embodiments, the pulse width may be less than 2 ms but greater than 1 μs.

In some embodiments, the distance between the first and second electrodes may be in a range of 1 nm and 10 nm.

In some embodiments, the one or more mental materials may include, but are not limited to, silver (Ag), nickel (Ni), cobalt (Co), Ni alloy, Co alloy, gold, palladium, platinum, iridium, or alloys thereof, or combinations thereof.

In some embodiments, the first electrode and the second electrode are orthogonal to the nano-fluidic channel and forms a self-aligned transverse tunneling junction with the nanogap on the planar substrate.

In some embodiments, the electromagnetic beam may be a laser beam.

In some embodiments, the laser beam may be an ultraviolet laser beam; in some embodiments, the laser beam may be a visible laser beam; in some embodiments, the laser beam may be an infrared laser beam.

In some embodiments, the optical coupling element may include a lens assembly, a fiber optical coupling element, a waveguide element, or a combination thereof.

In some embodiments, the optical coupling element may include a lens assembly.

In some embodiments, the optical coupling element may include a fiber optical coupling element such as a fiber-in-fiber-out coupling element.

In some embodiments, the optical coupling element may include a waveguide element.

In some embodiments, the optical coupling element may further include a polarizer.

In some embodiments, the optical signal is selected from the group consisting of a fluorescence or phosphorescence signal, a tip-enhanced Raman signal, a surface-enhanced Raman signal, and a combination thereof.

In some embodiments, the optical signal is a fluorescence or phosphorescence signal.

In some embodiments, the optical signal is a tip-enhanced Raman signal.

In some embodiments, the optical signal is a surface-enhanced Raman signal.

In some embodiments, the first electrode and second electrode may include an oxide layer such as a metal oxide layer.

In some embodiments, the first electrode and second electrode may include a surface modification layer including, but not limited to, polyethylene glycol thiol (PEG-thiol), alkyl thiol, cysteine, 4(5)-(2-mercaptoethyl)-1H-imidazole-2-carboxamide, or a combination thereof.

In some embodiments, the first electrode and second electrode may include a reader molecule, such as cysteine, 4(5)-(2-mercaptoethyl)-1H-imidazole-2-carboxamide.

In some embodiments, the biomolecule may include, but is not limited to, RNA, DNA, protein, peptide.

In some embodiments, the biomolecule may be RNA.

In some embodiments, the biomolecule may be DNA.

In some embodiments, the biomolecule may be peptide or protein.

In some embodiments, the first bias may be in a range of −5 mV to −1500 mV.

In some embodiments, the second bias may be in a range of −1000 mV to 1000 mV.

In some embodiments, the tunneling current, ionic current, and optical signal are originated from a coincidental and colocalized translocation event when the biomolecule translocates the nanogap.

In some embodiments, the method may further include providing a third bias between the third electrode and the fourth electrode across the nano-fluidic channel for a first period of time before providing the first bias.

In some embodiments, the method may further include adjusting a polarization direction of the electromagnetic beam relative to a transverse direction of the nanogap.

In some embodiments, the method may further include adjusting an ionic strength of the sample solution.

In some embodiments, the method may further include adding a translocation speed regulator in the sample solution, wherein the translocation speed regulator may be silica nanoparticles.

In some embodiments, the method may further include adjusting the distance between the first and second electrodes.

In some embodiments, the method further comprises adjusting a sharpness of the first and second electrodes

In some embodiment, the correlating the coincidental and colocalized tunneling current, the ionic current, and the optical signal to determine a sequence of the biomolecule comprises analyzing the coincidental and colocalized tunneling current, the ionic current, and the optical signal by utilizing a machine learning algorithm to determine the sequence of the biomolecule, wherein the machine learning algorithm may be a support vector machine.

Therefore, the nanopore-optical hybrid electronic device and the method for sequencing a biomolecule and modifications thereof disclosed herein integrate a self-aligned transverse tunneling junction with a nanopore on a planar substrate, which facilitates recording the optical characteristics of the biomolecule when the biomolecule translocates the nanopore. The transverse tunneling junction further provides strong and highly localized optical enhancements for the biomolecule translocating through the nanopore. The combined coincidental and colocalized tunneling current, ionic current, and enhanced optical signal provide a multi-dimensional signal space for accurately identifying different RNA modifications, peptide sequences/modifications, and other biomolecules. Through the analysis of the correlation of coincidental and colocalized tunneling current, ionic current, and enhanced optical signal such as Raman spectrum, the base and base modifications of RNA and/or DNA, and the amino acids and amino acid modifications of peptide/protein may be sequenced in a high yield and high accuracy, thereby achieving direct biomolecule sequencing.

Additional aspects will be set forth in part in the description which follows and, in part, will be apparent from the description, or may be learned by practice of the presented embodiments.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 illustrates a schematic diagram of a nanopore-optical electronic device according to some embodiments of the present disclosure.

FIG. 2 illustrates a reversible pulsed electrochemical deposition controlling nanogap and tunneling junction dimensions according to some embodiments of the present disclosure.

FIG. 3 illustrates an initial and final shape of transverse electrodes of a nanopore-optical electronic device according to some embodiments of the present disclosure.

FIGS. 4A-4D illustrate DNA translocation events detected simultaneously from ionic current and from tunneling current between transverse electrodes for a nanopore device according to some embodiments of the present disclosure. FIG. 4A illustrates correlated current spikes with consistent polarities recorded when lambda-DNA is introduced in the cis-chamber with a negative bias. FIG. 4B illustrates a magnified event taken from the record. FIG. 4C illustrates all events normalized by its magnified and aligned by the peak time of the ionic current. FIG. 4D illustrates images tracking DNA molecules stained by Sytox orange, Scale bar: 10 μm.

FIGS. 5A-5D illustrate characteristics of signals corresponding to forward/backward and uncorrelated/correlated translocation events for a nanopore-optical device according to some embodiments of the present disclosure. FIG. 5A illustrates the offset of peak time of the transverse tunneling current signals vs the ionic current signals. Negative value means leading in time. FIG. 5B illustrates the comparison of the full width at half maximum (FWHM) of current signal between correlated ionic and transverse tunneling current signals. FIG. 5C illustrates the comparison of FWHM for transverse tunneling current events that have correlated ionic events and those that do not. FIG. 5D illustrates the comparison of peak height of transverse tunneling current events that have correlated ionic events and those that do not.

FIGS. 6A-6D illustrate DNA enrichment in the nano-fluidic channel under a driving bias according to some embodiments of the present disclosure. FIG. 6A illustrates current events when DNA is just introduced at a low driving bias. FIG. 6B illustrates DNA concentration at the rim of the channel after applying a higher driving bias. FIG. 6C illustrates current events with increased rate after applying higher driving bias and then returning to the same low driving bias. FIG. 6D illustrates no events detected at the reversed driving bias.

FIGS. 7A-7B illustrate examples of DNA and amino acid binding to transverse electrodes of a nanopore device according to some embodiments of the present disclosure. FIG. 7A illustrates a self-matching DNA molecule that has thiol group modification at 3′. FIG. 7B illustrates L-cysteine.

FIGS. 8A-8B illustrate characteristic conductance traces after dsDNA forms bridges between transverse electrodes of a nanopore device according to some embodiments of the present disclosure. FIG. 8A illustrates 2.5 seconds of recording out of a half an hour experiment. FIG. 8B illustrates an expansion of the data from 4.9 seconds to 5.1 seconds in FIG. 8A.

FIGS. 9A-9B illustrate histogram of the conductance distribution. FIG. 9A illustrates the histogram of the conductance distribution with fitting using Gaussian peaks. FIG. 9B illustrates the fit conductance peak position plotted vs peak number.

FIGS. 10A-10B illustrate a reader molecule according to some embodiments of the present disclosure. FIG. 10A illustrates a binding mode with a ribonucleotide. FIG. 10B illustrates tautomerization of ICA.

FIGS. 11A-11C illustrate Raman signal obtained from transverse electrodes modified with L-cysteine in a nanopore device according to some embodiments of the present disclosure. FIG. 11A illustrates a bright field image of the nanopore device. FIG. 11B illustrates a Rayleigh scattering image of the nanogap. FIG. 11C illustrates Raman spectrum obtained from the nanogap (at cross position in FIG. 11B).

FIGS. 12A-12C illustrate calculated Raman spectra of nuclear bases according to some embodiments of the present disclosure. FIG. 12A illustrates structures of Adenosine, Cytidine, Guanosine, and Uridine. FIG. 12B illustrates structures of N⁶-methyladenosine and Inosine. FIG. 12C illustrates calculated Raman frequencies of nuclear bases shown in FIG. 12A and FIG. 12B.

### DETAILED DESCRIPTION

In the following detailed description, reference is made to the accompanying drawings that form a part hereof, and in which are shown by way of illustration embodiments that may be practiced. It is to be understood that other embodiments may be utilized, and structural or logical changes may be made without departing from the scope. Therefore, the following detailed description is not to be taken in a limiting sense, and the scope of embodiments is defined by the appended claims and their equivalents.

The terms used herein are only for the purpose of describing specific embodiments, and are not intended to limit of the disclosure. As used in this disclosure and the appended claims, the singular forms “a,” “an,” and “the” are intended to include the plural forms as well, unless the context clearly indicates otherwise. It should also be understood that the terms “or,” and “and/or” as used herein refers to and encompasses any or all possible combinations of one or more associated listed items. For example, a phrase in the form “A or B” or in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

It should be noted that in the instant disclosure, relational terms such as “first,” “second,” “third,” etc. are used herein merely to distinguish one entity or operation from another entity or operation without necessarily requiring or implying any such actual relationship or order between such entities or operations.

The term “about” may mean within plus or minus 10% of a stated value. For example, “about 100” may refer to any number between 90 and 110.

Any numerical range recited herein is intended to include all sub-ranges of the same numerical precision subsumed within the recited range. For example, a range of “1.0 to 10.0” or “between 1.0 and 10.0” is intended to include all 1 subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1-35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Unless otherwise noted, technical terms are used according to conventional usage. Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general references.

### I. Terms

To facilitate review of the various embodiments of the disclosure, the following explanations of specific terms are provided:

Analyte: A substance whose chemical constituents are being identified and measured. For example, an analyte may include RNA, DNA, peptides, proteins, enzymes, and/or other biomolecules which have an extremely small volume.

Binding or stable binding: An association between two substances or molecules, such as the association of an antibody with a peptide. Binding can be detected by any procedure known to one skilled in the art, such as by physical or functional properties of the formed complexes.

Biomolecule: A molecule that is produced by a living organism. For example, a biomolecule is an organic molecule and especially a macromolecule (such as a protein, a nucleic acid, a peptide) in living organisms. In some examples, biomolecule is interchangeable with “analyte molecule.”

Chemical Modification: A number of various processes involving the alteration of the chemical constitution or structure of molecules. In one example, a chemically modified electrode is an electrode that has a surface chemically converted to change the electrode's properties, such as its' physical, chemical, electrochemical, optical, electrical, and/or transport characteristics.

Cis-chamber and Trans-chamber: A “cis-chamber” is a first chamber, and a “trans-chamber” is a second chamber that is opposite to the cis-chamber, such as on an opposite side of cis-chamber. In embodiments, the cis chamber is a chamber with a negative electrode and the trans-chamber on the opposite side of a nanogap is the chamber with a positive electrode such that a negatively charged molecule in the cis-chamber may be guided through the nanogap to trans-chamber by a driving bias. In the present disclosure, term “cis-chamber” and term “cis-fluidic chamber” are interchangeable, and term “trans-chamber” and term “trans-fluidic chamber” are interchangeable. The cis-chamber and trans-chamber may have different shapes and dimensions and may have a plurality of sections having different shapes and dimension. For example, referring to FIG. 3, either or both of the trans-chamber and the cis-chamber may include one or more supporting walls 130 also operating as partitions forming a plurality of translocation guidance canals 135.

Contacting: Placement in direct physical association, including both a solid and liquid form.

Current: The term “current” may refer to the current signal generated over time from a device described herein. In the present disclosure, the term “current” and the term “current signal” are interchangeable, such as “tunneling current” is interchangeable with “tunneling current signal,” “ionic current” is interchangeable with “ionic current signal.”

Electrochemical Deposition: A process by which a thin and tightly adherent desired coating of metal, oxide, or salt can be deposited onto the surface of a conductor substrate by simple electrolysis of a solution containing the desired metal ion or its chemical complex. Electrochemical deposition transports metal ions in a solution by an electric field to coat the surface of a substrate. Electrochemical deposition is an efficient procedure to prepare metal nanoparticles.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” may be understood to have been purified by standard purification methods. The term also embraces nucleic acid molecules and proteins prepared by recombinant expression in a host cell as well as chemically synthesized nucleic acid molecules and proteins.

Label: An agent capable of detection, for example, a label can be attached to a nucleic acid molecule or protein (indirectly or directly), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, New York, 1989) and Ausubel et al. (In Current Protocols in Molecular Biology, John Wiley & Sons, New York, 1998).

Linked or linker: The term “linked” means joined together, either directly or indirectly. For example, a first moiety may be covalently or noncovalently (e.g., electrostatically) linked to a second moiety. This includes, but is not limited to, covalently bonding one molecule to another molecule, noncovalently bonding one molecule to another (e.g., electrostatically bonding), non-covalently bonding one molecule to another molecule by hydrogen bonding, non-covalently bonding one molecule to another molecule by van der Waals forces, and any and all combinations of such couplings. Indirect attachment is possible, such as by using a “linker” (a molecule or group of atoms positioned between two moieties).

In some embodiments, linked components are associated in a chemical or physical manner so that the components are not freely dispersible from one another. For example, two components may be covalently bound to one another so that the two components are incapable of separately dispersing or diffusing.

Nucleic acid: A deoxyribonucleotide or ribonucleotide polymer, which may include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. The term “nucleotide” refers to a base-sugar-phosphate combination and includes ribonucleoside triphosphates ATP, UTP, CTG, GTP and deoxyribonucleoside triphosphates such as dATP, dCTP, dITP, dUTP, dGTP, dTTP, or derivatives thereof.

Optional: “Optional” or “optionally” means that the subsequently described event or circumstance can but need not occur, and that the description includes instances where said event or circumstance occurs and instances where it does not.

Protein: The terms “protein,” “peptide,” “polypeptide” may refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Praline P Pro; Glutamine Q Gln; Arginine R Arg; Serine S Ser; Threonine T Thr; Valine V Val; Tryptophan w Trp; and Tyrosine Y Tyr. In one embodiment, a peptide is an antibody or fragment or part thereof, for example, any of the fragments or antibody chains listed above. In some embodiments, the peptide may be post-translationally modified.

Raman Spectroscopy: A spectroscopic technique typically used to determine vibrational modes of molecules, although rotational and other low-frequency modes of systems may also be observed. Raman spectroscopy can be used in chemistry to provide a structural fingerprint by which molecules can be identified.

Sample: A mixture of molecules that comprises at least an analyte molecule that is subjected to manipulation in accordance with the systems and/or methods of the disclosure.

Translocation: A change in location. As used herein, a DNA/RNA translocation event may refer to DNA/RNA moving through a nanogap.

Transparent substrate: A material made up of components with a uniform index of refraction. Transparent materials appear clear, with the overall appearance of one color, or any combination leading up to a brilliant spectrum of every color; light is allowed to pass through the substrate without appreciable scattering of light. The opposite property of translucency is opacity or non-transparent. Examples of transparent substrates may include, but are not limited to, glass or quartz.

### II. General Description

A “true” RNA sequencing solution may include several capabilities: single-molecule control, single-base resolution, and chemical accuracy. Although currently there may be no method that can satisfy all of them, a nanopore device seems to be a promising starting platform, which is based on the detection of ionic current fluctuations across a nanoscale orifice as individual RNA molecules are driven to pass through the confined space. This is a very good configuration to confine, manipulate and investigate a single molecule, but several challenges remain to be addressed. Specifically, the ionic current readout has a fundamental resolution limit of ˜3 nm due to the extension of electric field on either side of the pore being comparable to its diameter. To perform correct sequencing significant data processing to differentiate combinations of 4-5 bases, corresponding to 256˜1024 possible levels of signals, passing through the biological nanopores. Although in principle the modification of RNA bases may bring characteristic ionic current fluctuations too, the exponentially increasing signal combinations may overwhelm the power of data processing.

Recently alternative methods have been proposed for the solid-state nanopore systems to address the problem with ionic current detection. Specifically, the tunneling current across a pair of metal electrodes when a DNA/RNA molecule is sandwiched between them could potentially achieve much higher spatial resolution and chemical specificity. For example, the scanning tunneling microscope (STM) and mechanical break-junction (MBJ) have been used to study tunneling current signatures of the four nucleobases, highlighting the importance of accurate control of gap size, incorporation of recognition ligands, and statistical identification. However, precise alignment of fixed electrodes to a nanopore structure has proved very difficult, and results based on focused ion beam (FIB), electron-beam induced deposition (EBID), top-down lithography and electromigration have demonstrated very limited success. In addition, the analysis of the tunneling data also relies significantly on machine learning and recently a study on recognition tunneling of canonical and modified RNA nucleotides showed a low calling accuracy.

On the other hand, surface enhanced Raman scattering (SERS) or tip-enhanced Raman scattering (TERS) have recently observed great progress on detecting characteristic vibration modes of single molecules. Specifically, the target molecules can be trapped within metal nanogaps or cavities, created by a roughened metal surface, closely packed metal nanoparticles, or a sharp metal tip near a substrate. The highly focused electric field within the gap/cavity (electromagnetic enhancement), and the interaction of trapped molecules with the metal surface (chemical enhancement) can give many orders of magnitude increase of Raman scattering signals, which may otherwise be undetectable for a single molecule. For example, the SERS signals have been recorded for DNAs translocating inside a fractal array of plasmonic nanopores, and the SERS from a plasmonic nano-slit can give single-molecule Raman spectroscopic fingerprinting of four nucleobases. However, Raman measurements have been limited to quasi-static mapping or recording of random single-molecule events, possibly due to the difficulty in the integration with definite control of the position and motion of a single molecule.

Disclosed herein are systems and methods for the detection, sequencing, and manipulation of microscopic specimens, such as single molecules including biomolecules, single small particles, or small quantities of matter such as DNA/RNA, protein/peptide as the samples are passed through a nanoscale gap in a nanofluidic channel. In particular, disclosed are new systems and methods to reliably mount a single molecule between a pair of metal electrodes and to allow both electrical and optical characterization of the structure and dynamics of the molecule. The molecule mounting/translocating event can be simultaneously detected by the ionic current changes inside the nanofluidic chambers, the tunneling current across the metal nanogap, and optical characteristics of the molecule enhanced by the metal electrodes, providing unambiguous analysis of background free signals.

It may be advantageous to have a new design of device structure to precisely deliver and detect single molecules at a tunneling gap for unambiguous and reproducible characterization. The nanopore-optical electronic device and the method for sequencing a biomolecule and modifications thereof disclosed herein integrate a self-aligned transverse tunneling junction with a nanopore on a planar substrate, which facilitates recording the optical characteristics of a biomolecule when the biomolecule translocates the nanopore. The transverse tunneling junction further provides strong and highly localized optical enhancements for the biomolecule translocating through the nanopore. The combined coincidental and colocalized tunneling current, ionic current, and enhanced optical signal provide a multi-dimensional signal space for accurately identifying different RNA modifications, peptide sequences/modifications, and other biomolecules. Through the analysis of the correlation of coincidental and colocalized tunneling current, ionic current, and enhanced optical signal such as Raman spectrum, the base and base modifications of RNA and/or DNA, and the amino acids and amino acid modifications of peptide/protein may be sequenced in a high yield and high accuracy, thereby achieving direct biomolecule sequencing.

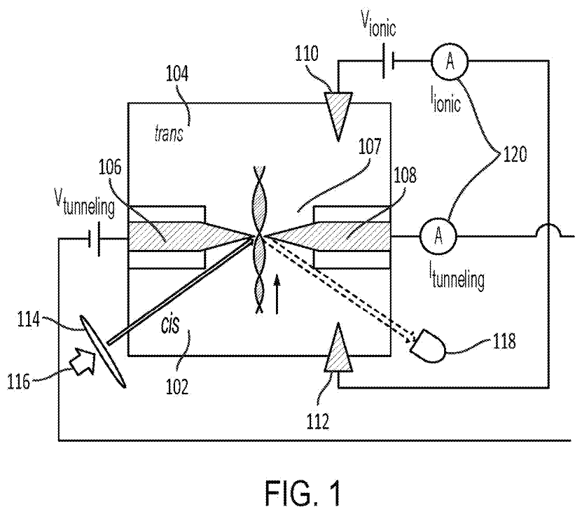

FIG. 1 illustrates a schematic diagram of a nanopore-optical electronic device according to some embodiments of the present disclosure. As shown in FIG. 1, the nanopore-optical electronic device may include:

a cis-fluidic chamber 102 and a trans-fluidic chamber 104 in a planar substrate;

a nano-fluidic channel 107 connecting the cis-fluidic chamber and the trans-fluidic chamber:

a first electrode 106 and second electrode 108 sealed in the nano-fluidic channel 107, the first electrode 106 and the second electrode 108 forming a nanogap between the first electrode 106 and the second electrode 108;

a third electrode 112 and fourth electrode 110 in the cis-fluidic chamber 102 and the trans-fluidic chamber 104, respectively;

an optical coupling element 114 configured to couple an electromagnetic beam 116 with the nanogap;

an optical detector 118 configured to detect an optical signal from the nanogap when a biomolecule translocates through the nanogap; and

a current-measuring circuit 120 configured to concurrently measure a tunneling current between the first electrode and the second electrode and an ionic current between the third electrode and the fourth electrode.

In the present disclosure, the term “nanogap” may refer to a nanogap formed between the first electrode 106 and the second electrode 108. When viewing in the direction of the translocating molecules' path, the nanogap is a nanopore; therefore, in the present disclosure, the nanogap and the nanopore are utilized interchangeably.

In some embodiments, the planar substrate may be a transparent substrate, such as glass or quartz. The transparent substrate may be transparent to the electromagnetic beam to facilitate the detection of the optical signal from the nanogap. However, embodiments of the present disclosure are not limited thereto.

In some embodiments, the planar substrate may be a non-transparent substrate, such as silicon coated with a layer of oxide. In this scenario, a transparent top cover may be provided to facilitate the detection of the optical signal from the nanogap. The transparent top cover may be made of polydimethylsiloxane (PDMS) or resin and be utilized to seal the chambers.

In some embodiments, the first electrode 106, second electrode 108, third electrode 112, and fourth electrode 110 may be independently formed of gold, palladium, platinum, silver, or combinations thereof. For example, in one embodiment, the first electrode 106, second electrode 108, third electrode 112, and fourth electrode 110 may be formed of gold or platinum. In another embodiment, the first electrode 106, second electrode 108, third electrode 112, and fourth electrode 110 may be independently formed of palladium. In yet another embodiment, the first electrode 106, second electrode 108, third electrode 112, and fourth electrode 110 may be formed of silver.

In some embodiments, the first electrode 106 and the second electrode 108 may be electrochemically deposited with one or more metal materials within the nano-fluidic channel 107 and under feed-back control, thereby forming the nanogap with a single path for the biomolecule to translocate from the cis-fluidic chamber 102 to the trans-fluidic chamber 104 and a distance between the first and second electrodes being between about 1-100 nm to form the nanogap which is self-aligned and has a narrowest bottleneck in the path between the cis-fluidic and trans-fluidic chambers.

In some embodiments, the first electrode 106 and second electrode 108 being electrochemically deposited with one or more metal materials within the nano-fluidic channel 107 and under feed-back control may include a pulsed electrochemical deposition operation with a pulse width of 50 ms or less and a rest period of about 2 seconds between pulses.

In some embodiments, the pulse width may be in a range of 1 μs-50 ms, 1 μs-40 ms, 1 μs-30 ms, 1 μs-20 ms, 1 μs-10 ms, 10 μs-10 ms, 20 μs-10 ms, 30 μs-10 ms, 40 μs-10 ms, 50 μs-10 ms, 60 μs-10 ms, 70 μs-10 ms, 80 μs-10 ms, 90 μs-10 ms, 100 μs-10 ms, 200 μs-10 ms, 300 μs-10 ms, 400 μs-10 ms, 500 μs-10 ms, 600 μs-10 ms, 700 μs-10 ms, 800 μs-10 ms, 900 μs-10 ms, 1 ms-10 ms, 2 ms-10 ms, 3 ms-10 ms, 4 ms-10 ms, 5 ms-10 ms, 6 ms-10 ms, 7 ms-10 ms, 8 ms-10 ms, or 9 ms-10 ms, or within any range defined between any two of the foregoing values, such as 10 μs-50 μs. In certain embodiment, the pulse width may be in a range of 1 μs-500 μs. In certain embodiments, the pulse width may be less than 2 ms but greater than or equal to 1 μs.

In some embodiments, the distance between the first electrode 106 and the second electrode 108 may be in a range of about 1-10 nm, about 2-10 nm, about 3-10 nm, about 4-10 nm, about 5-10 nm, about 6-10 nm, about 7-10 nm, about 8-10 nm, about 9-10 nm, about 10-20 nm, about 10-30 nm, about 10-40 nm, about 10-50 nm, about 10-60 nm, about 10-70 nm, about 10-80 nm, about 10-90 nm, or about 10-100 nm, or within any range defined between any two of the foregoing values, such as about 1-7 nm. In certain embodiments, the distance between the first electrode 106 and the second electrode 108 (i.e., the nanogap) may be in a range of about 1-10 nm. In certain embodiments, the distance between the first electrode 106 and the second electrode 108 (i.e., the nanogap) may be comparable to the diameter of a single biopolymer. For example, diameter of a single strand DNA or RNA molecule is about 1.0 nm. In certain embodiments, the largest size of the nanogap is comparable to the diameter of the three-dimensional size of a molecule, such as a protein molecule, which is to be characterized, such as on the order of about 5 nm to 10 nm, with larger ones close to 100 nm.

In some embodiments, the one or more mental materials may include, but are not limited to, silver (Ag), aluminum (Al), nickel (Ni), cobalt (Co), Ni alloy, Co alloy, gold, palladium, platinum, iridium, or alloys thereof, or combinations thereof.

In some embodiments, the first electrode 106 and the second electrode 108 are orthogonal to the nano-fluidic channel 107 and forms a self-aligned transverse tunneling junction with the nanogap on the planar substrate.

In some embodiments, the electromagnetic beam 116 may be a laser beam. The wavelength of the suitable laser beam may be in the wavelength region of ultraviolet spectrum (<400 nm) (e.g., an ultraviolet laser beam), in the wavelength region of visible spectrum (400-750 nm) (e.g., a visible laser beam), or in the wavelength region of near-infrared or infrared spectrum (>750 nm) (e.g., an infrared laser beam).

In some embodiments, the optical coupling element 114 may include a lens assembly such as a microscope objective lens.

In some embodiments, the optical coupling element 114 may include a fiber optical coupling element such as a fiber-in-fiber-out coupling element.

In some embodiments, the optical coupling element 114 may include a waveguide element.

In some embodiments, the optical coupling element 114 is configured to couple the laser beam at the center of the nanogap at a critical angle for total internal reflection imaging of the optical signals.

In some embodiments, the optical coupling element 114 may further include a polarizer. The polarizer may include, but is not limited to, an absorptive polarizer, a beam-splitting polarizer, birefringent polarizer, or a thin-film polarizer.

In some embodiments, the optical signal is selected from the group consisting of a fluorescence or phosphorescence signal, a tip-enhanced Raman signal, a surface-enhanced Raman signal, and a combination thereof.

In some embodiments, the optical signal is a fluorescence or phosphorescence signal.

In some embodiments, the optical signal is a tip-enhanced Raman signal.

In some embodiments, the optical signal is a surface-enhanced Raman signal.

In some embodiments, the first electrode 106 and second electrode 108 may include an oxide layer such as a metal oxide layer.

In some embodiments, the first electrode 106 and second electrode 108 may include a surface modification layer including, but not limited to, polyethylene glycol thiol (PEG-thiol), alkyl thiol, cysteine, 4(5)-(2-mercaptoethyl)-1H-imidazole-2-carboxamide, or a combination thereof.

In some embodiments, the first electrode 106 and second electrode 108 may include a reader molecule, such as cysteine, 4(5)-(2-mercaptoethyl)-1H-imidazole-2-carboxamide. The term “reader molecule” used herein may refer to a molecule that interacts with the first electrode and/or second electrode and an analyte to improve the tunneling current signal between the first electrode and the second electrode. In present disclosure, the reader molecule may also refer to as a “capture molecule” that may interact with an analyte such as a biomolecule through hydrogen bonding, electrostatic interactions, and/or aromatic π-π interactions.

In some embodiments, the biomolecule may include, but is not limited to, RNA, DNA, protein, peptide.

In some embodiments, the biomolecule may be RNA.

In some embodiments, the tunneling current, ionic current, and optical signal are coincidental in time and colocalized in space, and originate from the same biomolecule that translocates the nanogap.

The fabrication process of the nanopore chip may further refer to the PCT publication No. WO 2022/055604 A2, the entire content of which is incorporated herein by reference in its entirety.

The present disclosure further provides a method for a method for sequencing a biomolecule. The method may include the steps of:

providing a nanopore-optical electronic device including:
a cis-fluidic chamber and a trans-fluidic chamber in a planar substrate, a nano-fluidic channel connecting the cis-fluidic chamber and the trans-fluidic chamber,
a first electrode and a second electrode in the nano-fluidic channel, the first electrode and the second electrode forming a nanogap between the first electrode and the second electrode,
a third electrode and a fourth electrode in the cis-fluidic chamber and the trans-fluidic chamber, respectively,
an optical coupling element configured to couple an electromagnetic beam with the nanogap,
an optical detector configured to detect an optical signal from the nanogap when a biomolecule translocates through the nanogap, and
a current-measuring circuit configured to measure a tunneling current between the first electrode and the second electrode and an ionic current between the third electrode and the fourth electrode,

providing a sample solution including a biomolecule in the cis-fluidic chamber;

providing a first bias between the third electrode and the fourth electrode across the nano-fluidic channel;

providing a second bias across the first electrode and the second electrode across the nanogap;

concurrently measuring:
a tunneling current between the first electrode and the second electrode,
an ionic current between the third electrode and the fourth electrode when the biomolecule translocates through the nanopore, and
an optical signal from the nanogap when the biomolecule translocates through the nanopore; and

correlating the tunneling current, the ionic current, and the optical signal to determine a sequence of the biomolecule.

For detailed description of the nanopore-optical electronic device utilized by the method disclosed herein, the reference may be made to the above description, which will not be repeated for conciseness.

In the present disclosure, for the convenience of description, the third electrode 112 is disposed in the cis-fluidic chamber and is a negative electrode relative to the fourth electrode 110, and the fourth electrode 110 is disposed in the trans-fluidic chamber and is a positive electrode. When an analyte is DNA, RNA, or other biomolecule possessing negative charges, the first bias may be in a range of about −5 mV to −1500 mV, about −5 mV to −1400 mV, about −5 mV to −1300 mV, about −5 mV to −1200 mV, about −5 mV to −1100 mV, about −5 mV to −1000 mV, about −5 mV to −900 mV, about −5 mV to −800 mV, about −5 mV to −700 mV, about −5 mV to −600 mV, about −5 mV to −500 mV, about −5 mV to −400 mV, about −5 mV to −300 mV, about −5 mV to −200 mV, about −5 mV to −100 mV, about −5 mV to −90 mV, about −5 mV to −80 mV, about −5 mV to −70 mV, about −5 mV to −60 mV, about −5 mV to −50 mV, about −5 mV to −40 mV, about −5 mV to −30 mV, about −5 mV to −20 mV, or about −5 mV to −10 mV, or within any range defined between any two of the foregoing values, such as about −10 to −200 mV. In some embodiments, the first bias is in a range of about −5 mV to −200 mV. As such, the first bias is a driving bias to drive the analyte to translocate from the cis-fluidic chamber to the trans-fluidic chamber. The magnitude of the first bias may be adjusted to control the rate of translocation, or the sign of the first bias may be reversed to reverse the direction of the translocation. As such, it may be possible to use the first bias to (i) advance the biomolecule (or, if desired, move the biomolecule in the opposite direction) until a desired portion of the biomolecule is in the nanogap, and then (ii) hold the biomolecule in position while measurements of e.g., tunneling current and Rahman scattering are performed.

In some embodiments, the second bias may be in a range of about −1000 mV to 1000 mV, about −900 mV to 900 mV, about −800 mV to 800 mV, about −700 mV to 700 mV, about −600 mV to 600 mV, about −500 mV to 500 mV, about −400 mV to 400 mV, about −350 mV to 350 mV, about −300 mV to 300 mV, about −250 mV to 250 mV, about −200 mV to 200 mV, about −150 mV to 150 mV, about −100 mV to 100 mV, or about −50 mV to 50 mV. In some embodiments, the second bias may be in a range of about −200 mV to 200 mV.

In some embodiments, when the concentration of the biomolecule in the sample solution is low, the method may further include providing a third bias between the third electrode and the fourth electrode across the nano-fluidic channel for a first period of time before providing the first bias. The third bias may be in a range of about −5 mV to −1500 mV, about −5 mV to −1400 mV, about −5 mV to −1300 mV, about −5 mV to −1200 mV, about −5 mV to −1100 mV, about −5 mV to −1000 mV, about −5 mV to −900 mV, about −5 mV to −800 mV, about −5 mV to −700 mV, about −5 mV to −600 mV, about −5 mV to −500 mV, about −5 mV to −400 mV, about −5 mV to −300 mV, about −5 mV to −200 mV, about −5 mV to −100 mV, about −5 mV to −90 mV, about −5 mV to −80 mV, about −5 mV to −70 mV, about −5 mV to −60 mV, about −5 mV to −50 mV, about −5 mV to −40 mV, about −5 mV to −30 mV, about −5 mV to −20 mV, or about −5 mV to −10 mV, or within any range defined between any two of the foregoing values, such as about −500 to −1000 mV. In one embodiment, the third bias is about −1000 mV. The first period of time may be in a range of about 1 minute to 60 minutes, about 2 minutes to 60 minutes, about 3 minutes to 60 minutes, about 4 minutes to 60 minutes, about 5 minutes to 60 minutes, about 6 minutes to 60 minutes, about 7 minutes to 60 minutes, about 8 minutes to 60 minutes, about 9 minutes to 60 minutes, about 10 minutes to 60 minutes, about 20 minutes to 60 minutes, about 30 minutes to 60 minutes, about 40 minutes to 60 minutes, or about 50 minutes to 60 minutes, or within any range defined between any two of the foregoing values, such as about 2 minutes to 10 minutes.

In some embodiments, the method may further include adjusting a polarization direction of the electromagnetic beam relative to a transverse direction of the nanogap.

In some embodiments, the method may further include adjusting an ionic strength of the sample solution.

In some embodiments, the method may further include adding a translocation speed regulator in the cis-fluidic chamber and/or the trans-fluidic chamber. In certain embodiments, SERS/TERS signal may be collected at a time scale of about 10 ms, and recognition of tunneling signals may have a bandwidth of 1 MHz for a preamplifier of the current measuring circuit. So, the main bottleneck of data collection is the Raman spectroscopy. Therefore, a translocation speed regulator may be added to the sample solution to regulate the translocation speed of biomolecules. The translocation speed regulator may be silica nanoparticles with diameter of 10-20 nm. Silica nanoparticles may fill in the nano-fluidic channel by electrophoresis. The electroosmotic force may create a layer of closely packed colloid structure where the RNA molecules may diffuse through with much increased resistance. This could significantly slow down the translocation speed to match the speed of the recording of the nanopore-optical electronic device.

In some embodiments, the method may further include adjusting the distance between the first and second electrodes; in some embodiments, the method may further include adjusting a sharpness of the first and second electrodes. For example, through the pulsed electrochemical deposition described above by adjusting the pulse width, the rest period, and the one or more metal materials, the distance between the first and second electrodes and the sharpness (e.g., tip shapes) of the first electrode and the second electrode may be finely tuned.

In some embodiments, the correlating the tunneling current, the ionic current, and the optical signal to determine a sequence of the biomolecule may include analyzing the tunneling current, the ionic current, and the optical signal by utilizing a machine learning algorithm to determine a sequence of the biomolecule. For example, the machine learning algorithm may include a support vector machine (SVM).

The present disclosure further provides an integrated chip system including a plurality of the nanopore-optical electronic devices of the present disclosure for multiplexed detection.

The nanopore-optical hybrid electronic device and the method for sequencing a biomolecule and modifications thereof disclosed herein integrate a self-aligned transverse tunneling junction with a nanopore on a planar substrate, which facilitates recording the optical characteristics of the biomolecule when the biomolecule translocates the nanopore. The transverse tunneling junction further provides strong and highly localized optical enhancements for the biomolecule translocating through the nanopore. The combined coincidental and colocalized tunneling current, ionic current, and enhanced optical signal provide a multi-dimensional signal space for accurately identifying different RNA modifications, peptide sequences/modifications, and other biomolecules. Through the analysis of the correlation of coincidental and colocalized tunneling current, ionic current, and enhanced optical signal such as Raman spectrum, the base and base modifications of RNA and/or DNA, and the amino acids and amino acid modifications of peptide/protein may be sequenced in a high yield, thereby achieving direct biomolecule sequencing.

### EXAMPLES

Embodiments of the present disclosure will be further described by the following examples. The examples are illustrative and should not be interpreted as the limitations of the present disclosure.

### 1. Adjustment of the Nanogap Between Transverse Electrodes of the Nanopore-Optical Electronic Device

The nanogap between transverse electrodes (i.e., between the first electrode and the second electrode) of a nanopore-optical electronic device may be finely tuned and adjusted by utilizing feedback control techniques adopted from studies (Sadar, J.; Wang, Y.; Qing, Q., Confined Electrochemical Deposition in Sub-15 nm Space for Preparing Nanogap Electrodes. ECS Trans 2017, 77 (7), 65-72; Qing, Q.; Chen, F.; Li, P.; Tang, W.; Wu, Z.; Liu, Z., Finely tuning metallic nanogap size with electrodeposition by utilizing high frequency impedance in feedback. Angew Chem Int Ed Engl 2005, 44 (47), 7771-5), which are hereby incorporated by reference in their entireties.

The channels of the nanopore-optical electronic device were filled with electrolyte containing 18.5 mM KAu(CN)2 and 180 mM potassium citrate, and the conductance between the transverse electrodes was monitored in real time to control electrochemical deposition of Au onto the existing electrodes with Ag/AgCl serving as the counter electrode. The final dimension of the nanogap and the tunneling junction may be finely and reproducibly tuned with a reversible pulsed deposition strategy as shown below.

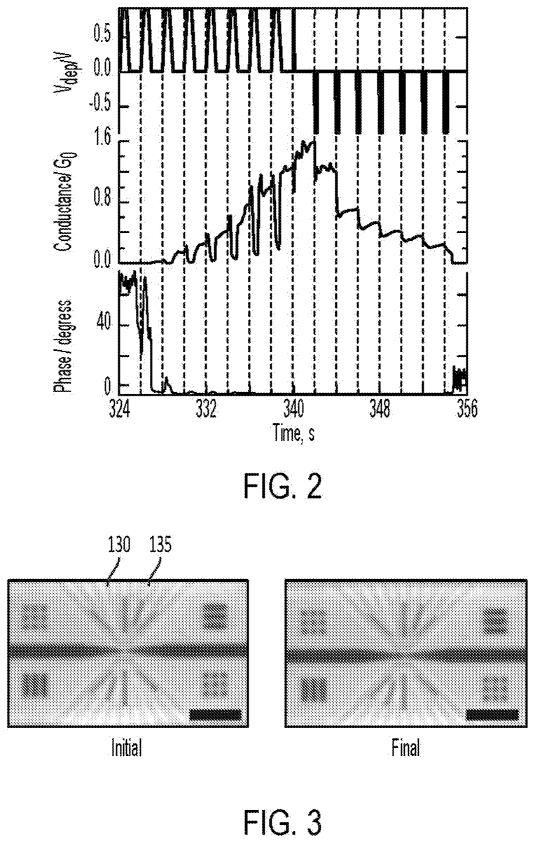

FIG. 2 illustrates a reversible pulsed electrochemical deposition controlling nanogap and tunneling junction dimensions according to some embodiments of the present disclosure. FIG. 3 illustrates an initial and final shape of transverse electrodes of a nanopore-optical electronic device according to some embodiments of the present disclosure. A unique advantage of using electrochemistry to tune the tunneling junction is that this process is fully reversible. A control program was created which can actively track the conductance across the transverse electrodes at the end of every period of the pulsatile deposition. If the conductance is above a threshold, the program will flip the polarity of the bias V_{dep} to switch from the deposition mode to dissolution mode such that Au is removed in controlled steps. FIG. 2 gives such an example where the transition happened at 342 s when the conductance reached above 1 conductance quantum G₀. More specifically, FIG. 2 depicts a record of the potential V_{dep} applied to the Ag/AgCl counter electrodes for controlled Au deposition (when V_{dep}>0) and dissolution (when V_{dep}<0), and the corresponding amplitude (in units of conductance quantum G₀=2·e²/h=77.5 μS) and phase of the AC conductance between the transverse electrodes recorded at 10.13 kHz. An automated control program monitored the conductance during the deposition. When the conductance reached a threshold above 1 G₀, the program switched the polarity of V_{dep} (at 342 sec) to remove Au from the electrode with negative pulses.

G₀ was used as the typical threshold as it can serve as the calibration of zero distance between the electrodes as they make first atomic contact. Such process can be repeated with high reproducibility to open and close the tunneling gap and generate a stable junction as defined by the final tunneling conductance.

It is herein noted that as the feedback control signal, the conductance between the transverse electrodes may be tracked using an AC signal ranging from I Hz to ˜10 kHz with a lock-in amplifier. For example, when higher frequency is used, the conductance may start showing clear changes at longer distance on the order of tens of nm due to the capacitive component, which gives wider range of control in distance. This is also demonstrated in the recorded phase of the AC conductance in FIG. 2 when 10.13 kHz reference signal was used, which showed an early transition in phase from more capacitive to more resistive before significant changes in the amplitude happened later. Nevertheless, in the present disclosure, dimensions are focused on that should best fit the diameter of the RNA/DNA molecules, therefore, low frequency AC signals (5 Hz) was used to monitor and control the junction's size which is most sensitive in the tunneling region. For devices used in RNA/DNA translocation experiments, the disclosed control program stabilizes the junction conductance to ˜1 nS, corresponding to a gap distance between 1-2 nm.

### 2. Coincidental Ionic and Tunneling Current Detection of Translocation Event

Lambda DNA solutions with different concentrations were prepared from commercial products (Sigma-Aldrich, SKU 10745782001) with filtered phosphate buffer (PB). During a DNA translocation test, the Lambda DNA solution was introduced into one microfluidic channel (cis channel) and the pure PB buffer was introduced into the other microfluidic channel (trans channel). One Ag/AgCl salt bridge reference applied a negative bias in the cis-chamber and the other Ag/AgCl salt bridge reference applied a constant 0 V in the trans-chamber. The transverse electrodes (i.e., pairs of the first electrode and the second electrode described above) applied desired gap bias (the second bias described above) perpendicular to the DNA translocation direction. The ionic current measurement between two microfluidic channels was performed by a patch clamp amplifier (HEKA patch clamp, Model #EPC 800), and the tunneling current measurement between the transverse electrodes was performed by a preamplifier (FEMTO, Model #: DLPCA-200).

When the nanopore and tunneling junctions were prepared, the chip was flushed by buffer PB, and the surface of the transverse electrodes formed of gold were modified with poly(ethylene glycol) methyl ether thiol (PEG thiol, Sigma-Aldrich) overnight to minimize the interaction between the DNA molecule and the Au surface. Lambda-DNA solution of 0.8 nM was prepared from commercial products (Sigma-Aldrich) with filtered PB and introduced into the cis-chamber, while the trans-chamber was filled only with PB. Ag/AgCl electrodes with salt bridge were used to apply a −50 m V bias for the cis-chamber and 0 V for the trans-chamber.

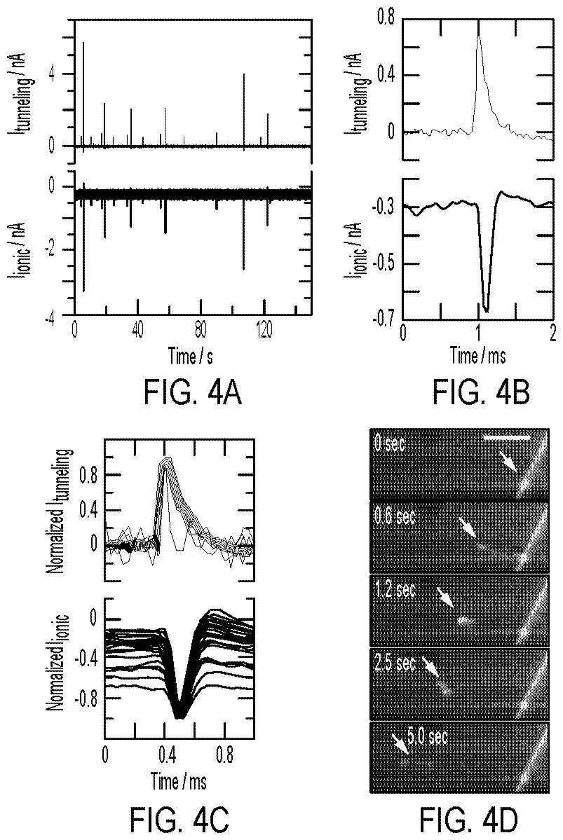

FIGS. 4A-4D illustrate DNA translocation events detected simultaneously from ionic current and from tunneling current between transverse electrodes for a nanopore device according to some embodiments of the present disclosure. When lambda-DNAs were introduced to the cis-chamber with a negative driving bias, within a few minutes the ionic current started showing characteristic negative spikes with an increase in magnitude, which is consistent with previous studies using low concentration electrolytes. Meanwhile, the current between the transverse electrodes showed coincidental positive spikes (see FIG. 4A). The ionic spikes typically showed a slight biphasic tail, while the spikes from the transverse electrodes demonstrated a slightly wider peak width with a longer tail and an offset in the peak time (see FIGS. 4B-4C). The fluorescently labelled DNA molecules were simultaneously tracked inside the confined nanofluidic channel by total internal reflection fluorescence imaging (see FIG. 4D). Lambda dsDNA introduced into the cis-chamber at a driving bias of −50 mV entered the nano-fluidic channels and moved at a speed of ˜5 μm/sec. The DNAs were stretched into linear shape due to the confined space (<200 nm).

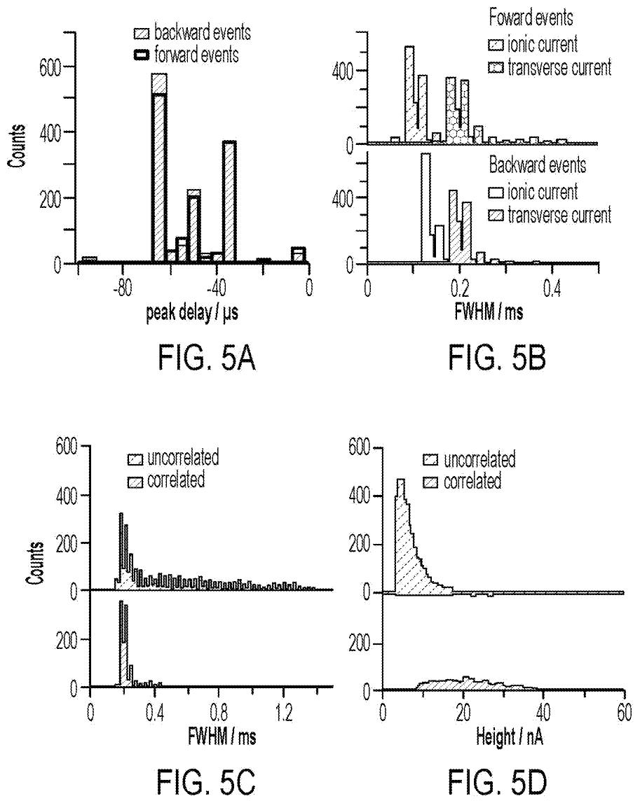

It is further noticed that, at very low driving bias (on the order of 10-20 mV), the “coincidental/correlated” ionic and tunneling events as well as “uncorrelated” events were observed. Notably, more than 93% ionic current spikes have correlated transverse current spikes with opposite polarity. On the other hand, >65% transverse current events did not have correlated ionic events. This contrasts with the data under higher driving bias where correlation in both readout signals was always observed. For correlated pairs, the overall distributions of peak height and width were similar (see FIG. 5D). The transverse tunneling current spikes demonstrated evenly spaced leading times of 35, 50 and 65 μs relative to the ionic current spikes. The transverse signals showed consistently wider peak width than the ionic signals. The high proportion of ionic events having correlated transverse tunneling signals suggests that the device has the precisely aligned the tunneling junction with the nanogap, such that almost all translocated molecules could interact with the transverse electrodes to generate signals.

Interestingly, about 65% of the transverse tunneling current signals at very low driving bias did not have correlated ionic spikes. Compared to the correlated tunneling signals, the uncorrelated signals showed consistently lower amplitude, but much broader distribution of peak width reaching up to 7 times wider (see FIG. 5C). The absence of coincidental ionic spikes indicated that the uncorrelated transverse tunneling signals come from DNA molecules that did not translocate across chambers but just moved between the electrodes. Therefore, through the analysis of the correlation of the coincidental/correlated ionic and tunneling events, the translocation of biomolecules may be identified with a high yield and high accuracy.

### 3. DNA Enrichment in the Nano-Fluidic Channel

When the concentration of an analyte is low, the concentration of the analyte may be increased by applying a large driving bias before data recording.

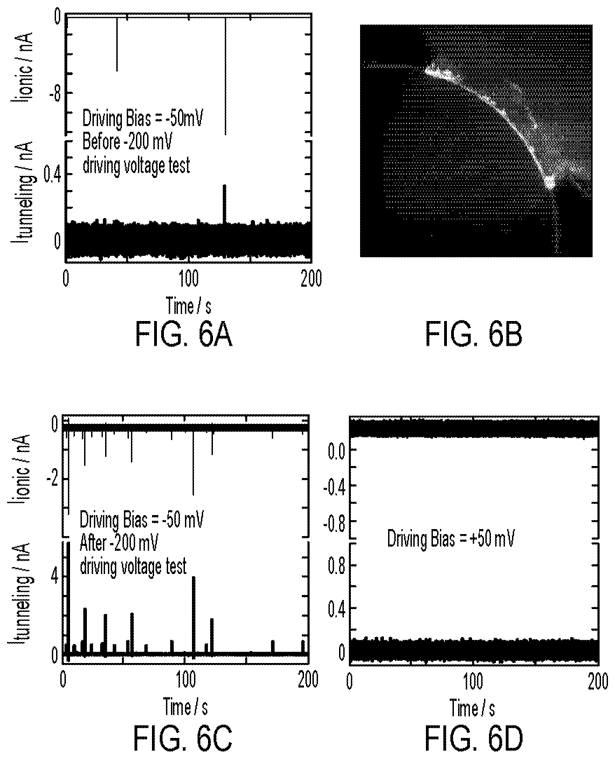

FIGS. 6A-6D illustrate DNA enrichment in the nano-fluidic channel under a driving bias according to some embodiments of the present disclosure. As shown in FIGS. 6A-6D, when lambda dsDNA was first introduced at low driving bias (−50 mV), the rate of current events was pretty low at 0.8 nM concentration (see FIG. 6A). After applying a higher driving bias (−200 mV), high concentration of DNA molecules was observed and enriched at the entry rim of the nano-fluidic channels (see FIG. 6B), and when return the same low driving bias (−50 mV) again, the rate of current events increased by an order of magnitude (see FIG. 6C). In addition, when the driving bias was set to positive when DNA was introduced, no events could be observed (see FIG. 6D).

### 4. Tunneling Conductance Measurement

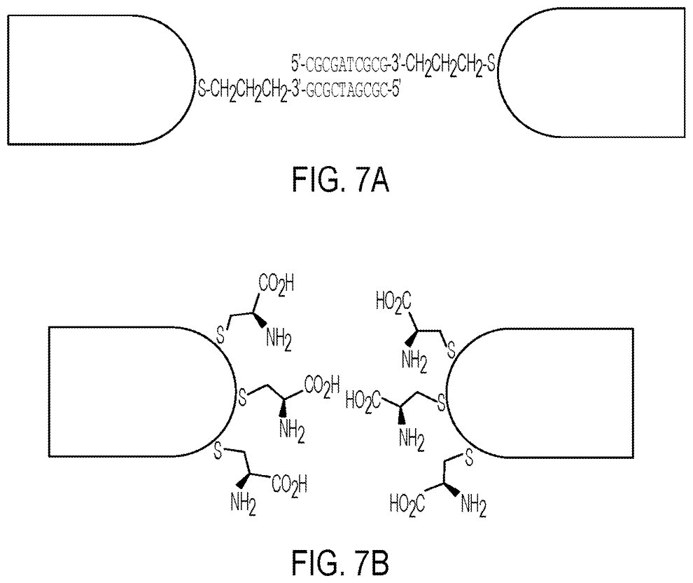

The tunneling conductance of biomolecules between transverse electrode is further investigated. FIGS. 7A-7B illustrate examples of DNA and amino acid binding to transverse electrodes (Q-NEP electrodes) of a nanopore device according to some embodiments of the present disclosure.

Specifically, after the tunneling conductance of the Q-NEP devices (i.e., the nanopore device) was stabilized to around 1 nS, the device was then flushed with buffer, followed by medium containing the target molecules at ˜1 uM concentration. In one device, a 10-base DNA molecule (5′-CGC GAT CGC G/3ThiolMC3-D/3′) that has a thiol group at the 3′ terminus was used. A double strand dimer by matching with its own sequence can be formed, so that after annealing the dsDNA will have thiol groups at both ends which can bridge the Q-NEP electrodes once introduced (see FIG. 7A). The DNA molecule bonds to the Au surface of transverse electrodes formed of gold (i.e., the first electrode and the second electrode) through an S—Au bond.

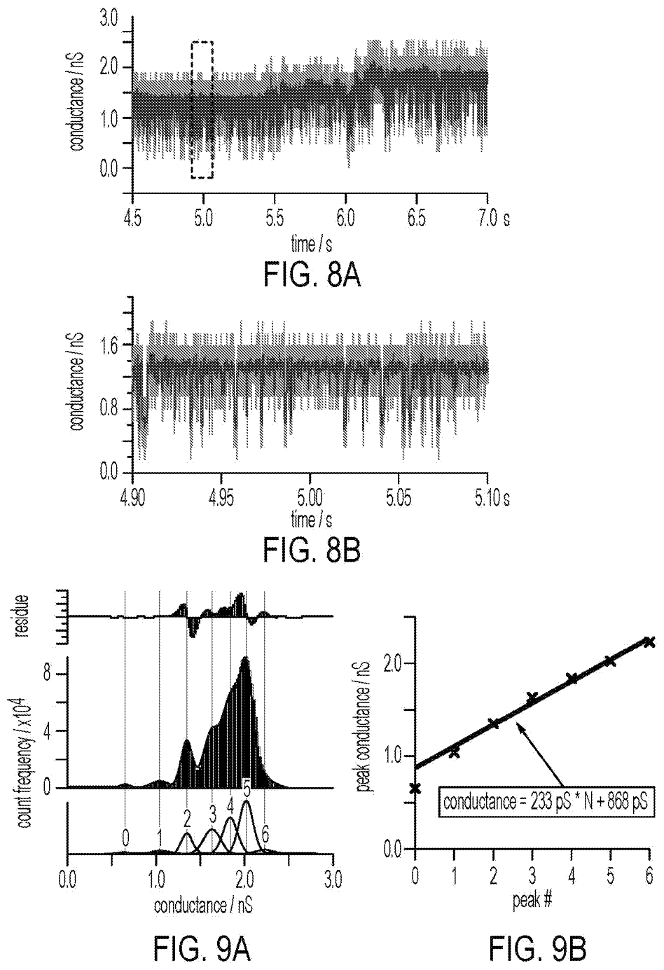

Please refer to FIGS. 8A-8B, which illustrate characteristic conductance traces after dsDNA forms bridges between transverse electrodes of a nanopore device according to some embodiments of the present disclosure. FIG. 8A illustrates 2.5 seconds of recording out of a half an hour experiment. FIG. 8B illustrates an expansion of the data from 4.9 seconds to 5.1 seconds in FIG. 8A. As shown in FIGS. 8A-8B, stable characteristic fluctuations in conductance between the Q-NEP electrodes were successfully observed with the 10-base dsDNA sample. The spike-like signals can be analyzed by histogram and the fitting of distribution. FIGS. 9A-9B illustrate an example of histogram of the conductance distribution. FIG. 9A shows the histogram of the conductance distribution with fitting using Gaussian peaks, and FIG. 9B shows the fit conductance peak position plotted vs peak number. The data analysis gives quantized values corresponding to 1-5 molecule bridges. The base tunneling conductance is 0.87 nS, with each molecule demonstrating 0.23 nS conductance.

### 5. Reader Molecule/Capture Molecule

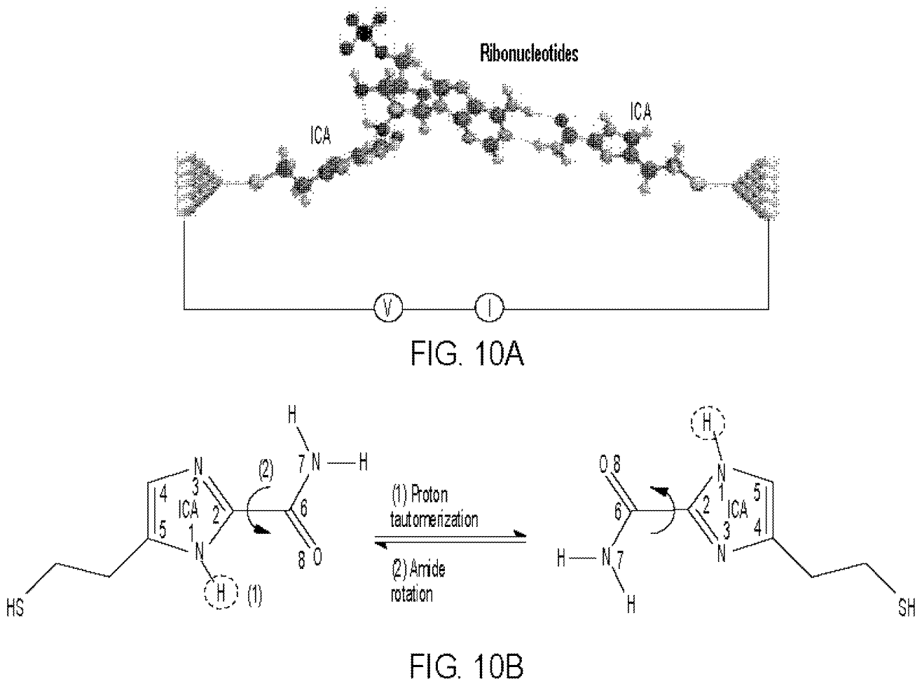

In one group of experiments, the transverse electrodes are modified with 4(5)-(2-mercaptoethyl)-1H-imidazole-2-carboxamide (ICA) molecules that have a structure with a rotatable amide connected to an imidazole ring for molecular recognition. ICA can capture a ribonucleotide by forming a hydrogen bonding triplex, which will produce optimal recognition tunneling signals for correlated analysis (see FIG. 10A). It has been demonstrated that ICA can capture four canonical and some based-modified ribonucleosides, such as Adenosine, Cytidine, Guanosine, Uridine, N⁶-methyladenosine, and Inosine, in a 2.5 nm nanogap, therefore ICA may facilitate to improve the tunneling current measurement and identify the type of ribonucleotides.

In another group of experiments, the transverse electrodes are modified with cysteine. Cysteine may capture a ribonucleotide by forming a hydrogen bonding complex, which will produce distinguishable tunneling signals for correlated analysis.

### 6. Raman Measurement

The nanopore chip is mounted on a Nikon Ti—U inverted microscope. The chip is bonded to a customized printed circuit board with integrated current preamplifiers and microfluidic control circuits for delivering fluidics to the channels on the chip. An excitation laser is projected at the center of the nanogap at a critical angle through a Nikon 60×TIRF oil objective lens for total internal reflection imaging of the Raman signal. The microscope is directly coupled to a Horiba iHR320 spectrometer through a set of relay lenses with a holographic notch that matches the laser. The output of the spectrometer will be recorded with an Andor EMCCD camera.

In one nanopore device, L-cysteine which has a thiol group was used to modify the Au surface of transverse electrodes formed of gold (i.e., the first electrode and the second electrode). L-cysteine bonds to the Au surface of transverse electrodes through an S—Au bond (see FIG. 7B).

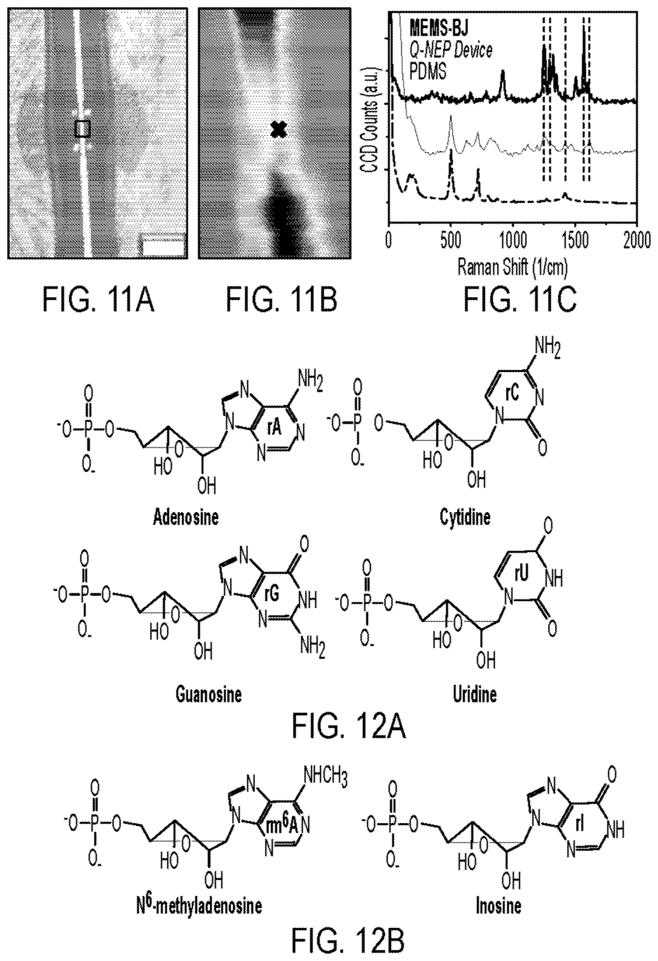

Please refer to FIGS. 11A-11C, which illustrate Raman signal obtained from transverse electrodes modified with L-cysteine in a nanopore device according to some embodiments of the present disclosure. FIG. 11A shows a bright field image of the nanopore device; FIG. 11B shows a Rayleigh scattering image of the nanogap and transverse electrodes framed in FIG. 11A; FIG. 11C shows Raman spectrum obtained from the nanogap (middle curve) at the cross position indicated in FIG. 11B. As shown in FIG. 11C, the tunneling-gap enhanced Raman spectrum from L-cysteine from the nanopore device was recorded. The peaks from the Raman spectrum (middle curve) showed background interference from PDMS sealings on the top of the chip, but had consistent characteristic peaks to L-cysteine Raman spectrum obtained with a break-junction setup (upper curve). In addition, from outside of the Q-NEP nanogap region, such peaks from L-cysteine were not detected, but only Raman peaks from PDMS were recorded (lower curve).

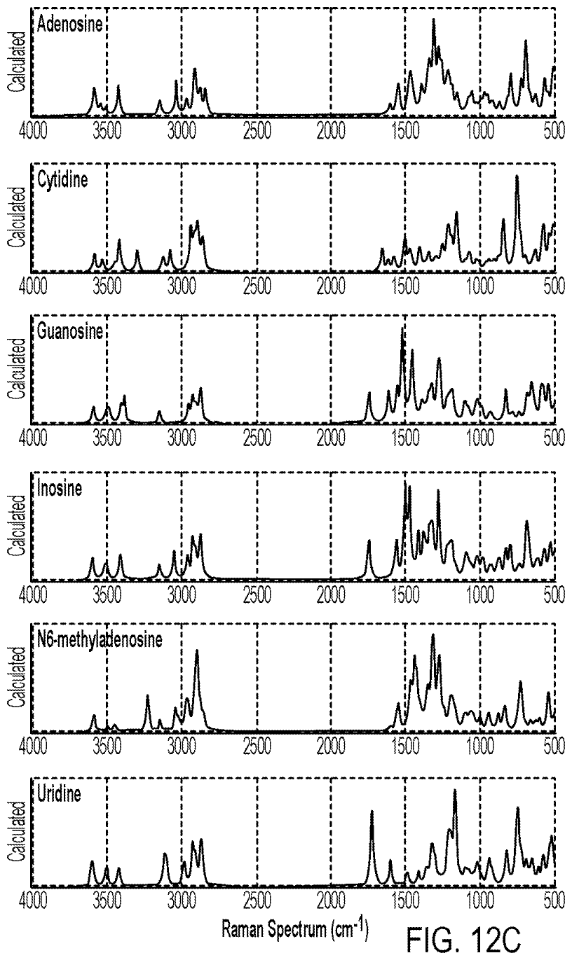

FIGS. 12A-12C illustrate calculated Raman spectra of nuclear bases according to some embodiments of the present disclosure. FIG. 12A illustrates structures of Adenosine, Cytidine, Guanosine, and Uridine; FIG. 12B illustrates structures of N⁶-methyladenosine and Inosine; FIG. 12C illustrates DFT calculated Raman spectra of nuclear bases shown in FIG. 12A and FIG. 12B. The calculated Raman spectra indicates that the nuclear bases and its modifications show their unique Raman frequencies, which can be advantageously utilized to identify the type of the nuclear bases.

The support vector machine (SVM) has been used to analyze recognition tunneling signals and nanopore data with high accuracy. For example, A set of recognition tunneling measurements could optimistically identify four RNA nucleosides with an accuracy of 92% on average and distinguish methylated adenosine (rm6A) and inosine from four RNA nucleosides with the accuracy of 85% and 78%, respectively, using a machine learning algorithm (such as a support vector machine, SVM). Therefore, the support vector machine (SVM) is further used to analyze the coincidental and colocalized tunneling current signal, the ionic current signal, and the tunneling-gap enhanced Raman signal to determine a sequence of the biomolecule with more improved accuracy.

### 7. Exemplary Embodiment

Some embodiments of the present disclosure may include features of the following numbered clauses:

Clause 1. A nanopore-optical electronic device, the nanopore-optical electronic device comprising:

a cis-fluidic chamber and a trans-fluidic chamber in a planar substrate;

a nano-fluidic channel connecting the cis-fluidic chamber and the trans-fluidic chamber;

a first electrode and a second electrode in the nano-fluidic channel, the first electrode and the second electrode forming a nanogap between the first electrode and the second electrode;

a third electrode and a fourth electrode in the cis-fluidic chamber and the trans-fluidic chamber, respectively;

an optical coupling element configured to couple an electromagnetic beam with the nanogap;

an optical detector configured to detect an optical signal from the nanogap when a biomolecule translocates through the nanogap; and

a current-measuring circuit configured to measure a tunneling current between the first electrode and the second electrode and an ionic current between the third electrode and the fourth electrode.

Clause 2. The nanopore-optical electronic device of clause 1, wherein the planar substrate is a transparent substrate.

Clause 3. The nanopore-optical electronic device of clause 2, wherein the transparent substrate is glass or quartz.

Clause 4. The nanopore-optical electronic device of clause 1, wherein the planar substrate is a non-transparent substrate.

Clause 5. The nanopore-optical electronic device of clause 4, wherein the non-transparent substrate is silicon coated with a layer of oxide.

Clause 6. The nanopore-optical electronic device of clause 1, wherein the first electrode, the second electrode, the third electrode, and the fourth electrode are independently formed of gold, palladium, platinum, silver, or combinations thereof.

Clause 7. The nanopore-optical electronic device of clause 6, wherein the first and second electrodes are electrochemically deposited with one or more metal materials within the nano-fluidic channel and under feed-back control, thereby forming the nanogap with a single path for the biomolecule to translocate from the cis-fluidic chamber to the trans-fluidic chamber, wherein:

the one or more mental materials comprise silver (Ag), nickel (Ni), cobalt (Co), Ni alloy, Co alloy, gold, palladium, platinum, iridium, or an alloy thereof, or a combination thereof,

a distance between the first and second electrodes is between 1 nm and 100 nm, and

the nanogap is self-aligned with the first and second electrodes and has a narrowest bottleneck in a path between the cis-fluidic chamber and the trans-fluidic chamber.

Clause 8. The nanopore-optical electronic device of clause 7, wherein the first and second electrodes being electrochemically deposited with one or more metal materials within the nano-fluidic channel and under feed-back control comprises a pulsed electrochemical deposition with a pulse width of 50 ms or less and a rest period of about 2 seconds between pulses.

Clause 9. The nanopore-optical electronic device of clause 8, wherein the pulse width is in a range of 2 ms to 5 ms.

Clause 10. The nanopore-optical electronic device of clause 8, wherein the pulse width is less than 2 ms but greater than 1 μs.

Clause 11. The nanopore-optical electronic device of clause 7, wherein the distance between the first and second electrodes is in a range of 1 nm to 10 nm.

Clause 12. The nanopore-optical electronic device of clause 7, wherein the first electrode and the second electrode are orthogonal to the nano-fluidic channel and forms a self-aligned transverse tunneling junction with the nanogap on the planar substrate.

Clause 13. The nanopore-optical electronic device of clause 1, wherein the electromagnetic beam comprises a laser beam.

Clause 14. The nanopore-optical electronic device of clause 13, wherein the optical coupling element comprises a lens assembly.

Clause 15. The nanopore-optical electronic device of clause 13, wherein the optical coupling element comprises a fiber optical coupling element.

Clause 16. The nanopore-optical electronic device of clause 13, wherein the optical coupling element comprises a waveguide element.

Clause 17. The nanopore-optical electronic device of any one of clauses 14-16, wherein the optical coupling element further comprises a polarizer.

Clause 18. The nanopore-optical electronic device of clause 13, wherein the optical signal is a fluorescence or phosphorescence signal.

Clause 19. The nanopore-optical electronic device of clause 13, wherein the optical signal is a tip-enhanced Raman signal.

Clause 20. The nanopore-optical electronic device of clause 13, wherein the optical signal is a surface-enhanced Raman signal.

Clause 21. The nanopore-optical electronic device of clause 1, wherein the first electrode and the second electrode further comprise a metal oxide layer.

Clause 22. The nanopore-optical electronic device of clause 1, wherein the first electrode and the second electrode further comprise a surface modification layer including polyethylene glycol thiol (PEG-thiol), alkyl thiol, cysteine, 4(5)-(2-mercaptoethyl)-1H-imidazole-2-carboxamide, or a combination thereof.

Clause 23. The nanopore-optical electronic device of clause 1, wherein the first electrode and the second electrode comprise a reader molecule including cysteine and/or 4(5)-(2-mercaptoethyl)-1H-imidazole-2-carboxamide.

Clause 24. The nanopore-optical electronic device of clause 1, wherein the biomolecule comprises RNA, DNA, protein, peptide, or combinations thereof.

Clause 25. The nanopore-optical electronic device of clause 24, wherein the biomolecule is RNA.

Clause 26. The nanopore-optical electronic device of clause 13, wherein the laser beam is an ultraviolet laser beam.

Clause 27. The nanopore-optical electronic device of clause 13, wherein the laser beam is a visible laser beam.

Clause 28. The nanopore-optical electronic device of clause 13, wherein the laser beam is an infrared laser beam.

Clause 29. The nanopore-optical electronic device of clause 1, wherein the tunneling current, the ionic current, and the optical signal are originated from a coincidental and colocalized translocation event when the biomolecule translocates the nanogap.

Clause 30. A method for sequencing a biomolecule, the method comprising:

providing a nanopore-optical electronic device comprising:
a cis-fluidic chamber and a trans-fluidic chamber in a planar substrate,
a nano-fluidic channel connecting the cis-fluidic chamber and the trans-fluidic chamber,
a first electrode and a second electrode in the nano-fluidic channel, the first electrode and the second electrode forming a nanogap between the first electrode and the second electrode,
a third electrode and a fourth electrode in the cis-fluidic chamber and the trans-fluidic chamber, respectively,
an optical coupling element configured to couple an electromagnetic beam with the nanogap,
an optical detector configured to detect an optical signal from the nanogap when a biomolecule translocates through the nanogap, and
a current-measuring circuit configured to measure a tunneling current between the first electrode and the second electrode and an ionic current between the third electrode and the fourth electrode;

providing a sample solution comprising a biomolecule in the cis-fluidic chamber;

providing a first bias between the third electrode and the fourth electrode across the nano-fluidic channel;

providing a second bias across the first electrode and the second electrode across the nanogap;

concurrently measuring:
a tunneling current between the first electrode and the second electrode,
an ionic current between the third electrode and the fourth electrode when the biomolecule translocates through the nanogap, and
an optical signal from the nanogap when the biomolecule translocates through the nanogap; and

correlating the tunneling current, the ionic current, and the optical signal to determine a sequence of the biomolecule.

Clause 31. The method of clause 30, wherein the planar substrate is a transparent substrate.

Clause 32. The method of clause 31, wherein the transparent substrate is glass or quartz.

Clause 33. The method of clause 30, wherein the planar substrate is a non-transparent substrate.

Clause 34. The method of clause 33, wherein the non-transparent substrate is silicon coated with a layer of oxide.

Clause 35. The method of clause 30, wherein the first electrode, the second electrode, the third electrode, and the fourth electrode may be formed of gold, palladium, platinum, silver, or combinations thereof.

Clause 36. The method of clause 30, wherein the first and second electrodes are electrochemically deposited with one or more metal materials within the nano-fluidic channel and under feed-back control, thereby forming the nanogap with a single path for the biomolecule to translocate from the cis-fluidic chamber to the trans-fluidic chamber, wherein:

the one or more mental materials comprise silver (Ag), nickel (Ni), cobalt (Co), Ni alloy, Co alloy, gold, palladium, platinum, iridium, or an alloy thereof, or a combination thereof,

a distance between the first and second electrodes is between 1 nm and 100 nm, and

the nanogap is self-aligned with the first and second electrodes and has a narrowest bottleneck in a path between the cis-fluidic chamber and the trans-fluidic chamber.

Clause 37. The method of clause 36, wherein the first and second electrodes being electrochemically deposited with one or more metal materials within the nano-fluidic channel and under feed-back control comprises a pulsed electrochemical deposition with a pulse width of 50 ms or less and a rest period of about 2 seconds between pulses.

Clause 38. The method of clause 37, wherein the pulse width is in a range of 2 ms to 5 ms.

Clause 39. The method of clause 37, wherein the pulse width is less than 2 ms but greater than 1 μs.

Clause 40. The method of clause 36, wherein the distance between the first and second electrodes is in a range of 1 nm to 10 nm.

Clause 41. The method of clause 36, wherein the first electrode and the second electrode are orthogonal to the nano-fluidic channel and forms a self-aligned transverse tunneling junction with the nanogap on the planar substrate.

Clause 42. The method of clause 30, wherein the electromagnetic beam comprises a laser beam.

Clause 43. The method of clause 30, wherein the optical coupling element comprises a lens assembly.

Clause 44. The method of clause 30, wherein the optical coupling element comprises a fiber optical coupling element.

Clause 45. The method of clause 30, wherein the optical coupling element comprises a waveguide element.

Clause 46. The method of any one of clauses 43-45, wherein the optical coupling element further comprises a polarizer.

Clause 47. The method of clause 30, wherein the optical signal is a fluorescence or phosphorescence signal.

Clause 48. The method of clause 30, wherein the optical signal is a tip-enhanced Raman signal.

Clause 49. The method of clause 30, wherein the optical signal is a surface-enhanced Raman signal.

Clause 50. The method of clause 30, wherein the first electrode and the second electrode further comprise a metal oxide layer.

Clause 51. The method of clause 30, wherein the first electrode and the second electrode further comprise a surface modification layer including polyethylene glycol thiol (PEG-thiol), alkyl thiol, cysteine, 4(5)-(2-mercaptoethyl)-1H-imidazole-2-carboxamide, or a combination thereof.

Clause 52. The method of clause 30, wherein the first electrode and the second electrode comprise a reader molecule including cysteine and/or 4(5)-(2-mercaptoethyl)-1H-imidazole-2-carboxamide.

Clause 53. The method of clause 30, wherein the biomolecule comprises RNA, DNA, protein, peptide, or combinations thereof.

Clause 54. The method of clause 53, wherein the biomolecule is RNA.

Clause 55. The method of clause 42, wherein the laser beam is an ultraviolet laser beam.

Clause 56. The method of clause 42, wherein the laser beam is a visible laser beam.

Clause 57. The method of clause 42, wherein the laser beam is an infrared laser beam.

Clause 58. The method of clause 30, wherein the tunneling current, the ionic current, and the optical signal are originated from a coincidental and colocalized translocation event when the biomolecule translocates the nanogap.

Clause 59. The method of clause 30, wherein the first bias is in a range of −5 mV to −1500 mV.

Clause 60. The method of clause 30, wherein the second bias is in a range of −1000 mV to 1000 mV.

Clause 61. The method of clause 30, wherein the method further comprises providing a third bias between the third electrode and the fourth electrode across the nano-fluidic channel for a first period of time before providing the first bias.

Clause 62. The method of clause 30, wherein the method further comprises adjusting a polarization direction of the electromagnetic beam relative to a transverse direction of the nanogap.

Clause 63. The method of clause 30, wherein the method further comprises adjusting an ionic strength of the sample solution.

Clause 64. The method of clause 30, wherein the method further comprises adding a translocation speed regulator in the sample solution, wherein the translocation speed regulator is silica nanoparticles.

Clause 65. The method of clause 36, wherein the method further comprises adjusting the distance between the first and second electrodes.

Clause 66. The method of clause 65, wherein the method further comprises adjusting a sharpness of the first and second electrodes.

Clause 67. The method of clause 30, wherein the correlating the tunneling current, the ionic current, and the optical signal to determine a sequence of the biomolecule comprises analyzing the tunneling current, the ionic current, and the optical signal by utilizing a machine learning algorithm to determine the sequence of the biomolecule, wherein the machine learning algorithm is a support vector machine.

The present disclosure can be embodied in-part in the form of computer implemented processes and apparatuses for practicing those processes. The present disclosure can also be embodied in-part in the form of computer program code containing instructions embodied in tangible media or computer readable storage medium, wherein, when the computer program code is loaded into, and executed by, an electronic device such as a computer, micro-processor or logic circuit, the device becomes an apparatus for practicing the present disclosure.

The present disclosure can also be embodied in-part in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the present disclosure. When implemented in a general-purpose microprocessor, the computer program code segments configure the microprocessor to create specific logic circuits.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A nanopore-optical electronic device, the nanopore-optical electronic device comprising:
a cis-fluidic chamber and a trans-fluidic chamber in a planar substrate;
a nano-fluidic channel connecting the cis-fluidic chamber and the trans-fluidic chamber;
a first electrode and a second electrode in the nano-fluidic channel, the first electrode and the second electrode forming a nanogap between the first electrode and the second electrode;
a third electrode and a fourth electrode in the cis-fluidic chamber and the trans-fluidic chamber, respectively;
an optical coupling element configured to couple an electromagnetic beam with the nanogap;
an optical detector configured to detect an optical signal from the nanogap when a biomolecule translocates through the nanogap; and
a current-measuring circuit configured to measure a tunneling current between the first electrode and the second electrode and an ionic current between the third electrode and the fourth electrode.

2. The nanopore-optical electronic device of claim 1, wherein the first electrode, the second electrode, the third electrode, and the fourth electrode are independently formed of gold, palladium, platinum, silver, or combinations thereof.

3. The nanopore-optical electronic device of claim 2, wherein the first and second electrodes are electrochemically deposited with one or more metal materials within the nano-fluidic channel and under feed-back control, thereby forming the nanogap with a single path for the biomolecule to translocate from the cis-fluidic chamber to the trans-fluidic chamber, wherein:
the one or more mental materials comprise silver (Ag), nickel (Ni), cobalt (Co), Ni alloy, Co alloy, gold, palladium, platinum, iridium, or an alloy thereof, or a combination thereof,
a distance between the first and second electrodes is between 1 nm and 100 nm, and
the nanogap is self-aligned with the first and second electrodes and has a narrowest bottleneck in a path between the cis-fluidic chamber and the trans-fluidic chamber.

4. The nanopore-optical electronic device of claim 3, wherein the first and second electrodes being electrochemically deposited with one or more metal materials within the nano-fluidic channel and under feed-back control comprises a pulsed electrochemical deposition with a pulse width of 50 ms or less and a rest period of about 2 seconds between pulses.

5. The nanopore-optical electronic device of claim 3, wherein the first electrode and the second electrode are orthogonal to the nano-fluidic channel and forms a self-aligned transverse tunneling junction with the nanogap on the planar substrate.

6. The nanopore-optical electronic device of claim 1, wherein the optical coupling element comprises a lens assembly, a fiber optical coupling element, a waveguide element, or a combination thereof.

7. The nanopore-optical electronic device of claim 6, wherein the optical coupling element further comprises a polarizer.

8. The nanopore-optical electronic device of claim 1, wherein the optical signal is selected from the group consisting of a fluorescence or phosphorescence signal, a tip-enhanced Raman signal, a surface-enhanced Raman signal, and a combination thereof.

9. The nanopore-optical electronic device of claim 1, wherein the first electrode and the second electrode further comprise a surface modification layer including polyethylene glycol thiol (PEG-thiol), alkyl thiol, cysteine, 4(5)-(2-mercaptoethyl)-1H-imidazole-2-carboxamide, or a combination thereof.

10. The nanopore-optical electronic device of claim 1, wherein the biomolecule comprises RNA, DNA, protein, peptide, or combinations thereof.

11. The nanopore-optical electronic device of claim 1, wherein the tunneling current, the ionic current, and the optical signal are coincidental in time and colocalized in space, and originate from the same biomolecule that translocates the nanogap.

12. A method for sequencing a biomolecule, the method comprising:
providing a nanopore-optical electronic device comprising:
a cis-fluidic chamber and a trans-fluidic chamber in a planar substrate,
a nano-fluidic channel connecting the cis-fluidic chamber and the trans-fluidic chamber,
a first electrode and a second electrode in the nano-fluidic channel, the first electrode and the second electrode forming a nanogap between the first electrode and the second electrode,
a third electrode and a fourth electrode in the cis-fluidic chamber and the trans-fluidic chamber, respectively,
an optical coupling element configured to couple an electromagnetic beam with the nanogap,
an optical detector configured to detect an optical signal from the nanogap when a biomolecule translocates through the nanogap, and
a current-measuring circuit configured to measure a tunneling current between the first electrode and the second electrode and an ionic current between the third electrode and the fourth electrode;
providing a sample solution comprising a biomolecule in the cis-fluidic chamber;
providing a first bias between the third electrode and the fourth electrode across the nano-fluidic channel;
providing a second bias across the first electrode and the second electrode across the nanogap;
concurrently measuring:
a tunneling current between the first electrode and the second electrode,
an ionic current between the third electrode and the fourth electrode when the biomolecule translocates through the nanogap, and
an optical signal from the nanogap when the biomolecule translocates through the nanogap; and
correlating the tunneling current, the ionic current, and the optical signal to determine a sequence of the biomolecule.

13. The method of claim 12, wherein the first and second electrodes are electrochemically deposited with one or more metal materials within the nano-fluidic channel and under feed-back control, thereby forming the nanogap with a single path for the biomolecule to translocate from the cis-fluidic chamber to the trans-fluidic chamber, wherein:
the one or more mental materials comprise silver (Ag), nickel (Ni), cobalt (Co), Ni alloy, Co alloy, gold, palladium, platinum, iridium, or an alloy thereof, or a combination thereof,
a distance between the first and second electrodes is between 1 nm and 100 nm, and
the nanogap is self-aligned with the first and second electrodes and has a narrowest bottleneck in a path between the cis-fluidic chamber and the trans-fluidic chamber.

14. The method of claim 12, wherein the optical coupling element comprises a lens assembly, a fiber optical coupling element, a waveguide element, or a combination thereof.

15. The method of claim 14, wherein the optical coupling element further comprises a polarizer.

16. The method of claim 12, wherein the optical signal is selected from the group consisting of a fluorescence or phosphorescence signal, a tip-enhanced Raman signal, a surface-enhanced Raman signal, and a combination thereof.

17. The method of claim 12, wherein the first electrode and the second electrode further comprise a surface modification layer including polyethylene glycol thiol (PEG-thiol), alkyl thiol, cysteine, 4(5)-(2-mercaptoethyl)-1H-imidazole-2-carboxamide, or a combination thereof.

18. The method of claim 12, wherein the biomolecule comprises RNA, DNA, protein, peptide, or combinations thereof.

19. The method of claim 12, wherein the tunneling current, the ionic current, and the optical signal are coincidental in time and colocalized in space, and originate from the same biomolecule that translocates the nanogap.

20. The method of claim 12, wherein the correlating the tunneling current, the ionic current, and the optical signal to determine a sequence of the biomolecule comprises analyzing the tunneling current, the ionic current, and the optical signal by utilizing a machine learning algorithm to determine the sequence of the biomolecule, wherein the machine learning algorithm is a support vector machine.
